(19) United States
(12) Patent Application Publication
(10) Pub. No.: US 2014/0053835 A1
(43) Pub. Date: Feb. 27, 2014

(54) GAS DISPENSER WITH DIFFUSING NOSEPIECE

(71) Applicant: Capnia, Inc., (US)

(72) Inventors: Scott Jay GILBERT, Menlo Park, CA (US); Pedro DE LA SERNA, San Jose, CA (US); Jay WILSON, Santa Cruz, CA (US)

(73) Assignee: Capnia, Inc., Palo Alto, CA (US)

(21) Appl. No.: 13/768,882

(22) Filed: Feb. 15, 2013

Related U.S. Application Data
(60) Provisional application No. 61/599,735, filed on Feb. 16, 2012.

(51) Int. Cl. A61M 15/08; A61M 16/12

(52) U.S. Cl. CPC A61M 15/08; A61M 16/12
USPC 128/203.14; 128/207.18

(57) ABSTRACT

Described here are hand-held, low flow devices for dispensing a therapeutic gas. The devices may be configured to include a gas control assembly for delivering a defined volume of gas at a controlled pressure and flow rate. A nosepiece may be included in the device that is formed of a porous material capable of filtering the dispensed gas, and also diffusing the flow of gas as it travels through the nosepiece and into the nasal cavity. The nosepiece may be configured so that there is substantially no restriction of flow therethrough. Methods for treating various medical conditions and delivering therapeutic gases to the nasal mucosa using hand-held, low flow gas dispenser devices are also described.

### CROSS-REFERENCE TO RELATED APPLICATIONS

This application claims priority to U.S. Provisional Application Ser. No. 61/599,735 filed Feb. 16, 2012, which is hereby incorporated by reference in its entirety.

### FIELD

Described here are hand-held, low flow devices for dispensing a therapeutic gas. The devices generally include a gas control assembly and a nosepiece that is formed of a porous material capable of simultaneously diffusing and filtering the flow of gas as it travels through the nosepiece and into the nasal cavity. Methods for delivering therapeutic gases using the hand-held, low flow devices are also described.

### BACKGROUND

Headaches, allergies and asthma are common medical conditions for which there is widespread interest in developing symptomatic treatment. Commercially available therapies include oral medicines, nasal sprays, oral inhalers, nasal inhalers, eye drops, and nose drops. Other possible therapies are available from the pharmacy with a prescription from a patient's doctor (e.g., injectables and inhalables). Despite the very large number of therapies which are available, no one therapy meets all patient needs, and many of the therapies suffer from significant shortcomings. For example, current therapies may be slow-acting, have numerous adverse side effects (e.g., nausea, drowsiness, rebound headache from analgesic overuse, rebound congestion from decongestant overuse, dizziness, sedation, addiction, and numerous others), have low efficacy, or are contraindicated for a large portion of patients (e.g., those with hypertension, coronary artery disease, cerebrovascular disease, peptic ulcers, pregnancy, concurrent medications that would interact, children, elderly, and others).

The use of diluted carbon dioxide by inhalation for treating symptoms related to medical conditions such as headaches, allergies, asthma, and nervous disorders was demonstrated in the 1940's and 1950's. The treatment protocols generally relied on breathing masks or other equipment for delivering relatively large volumes of dilute carbon dioxide to the patient for inhalation through the mouth and/or the nose into the lungs until they became unconscious. The efficacy of this treatment generally depended upon the systemic effects of the inhaled gas and therefore required large volumes of gas. Typical carbon dioxide volumes that were inhaled ranged from 0.5 to 25 liters of 30% to 70% carbon dioxide diluted in oxygen during a single treatment, which was repeated several times a week for 25 to 50 treatments. While the use of inhaled carbon dioxide has proven to be quite effective for a number of indications, the use of carbon dioxide delivered in this manner has never become a widely accepted practice. This may be the case because the method is limited by the necessity of making the patient unconscious, the length of the treatment time and course, the necessarily large, bulky non-portable gas cylinders, and the physician administration it requires. Most conventional systems are so large and heavy that they must be wheeled about using a dolly or a cart, and thus do not lend themselves to use outside of the hospital or home.

While hand-held carbon dioxide dispensers have been proposed, some are still designed to deliver large volumes of dilute carbon dioxide for inhalation. Other hand-held dispensers configured to provide low gas flow rates between about 0.5 cc/sec to about 20 cc/sec can also still be uncomfortable for the patient (e.g., the delivered gas creates an unpleasant stinging or burning sensation of the nasal mucosa), or require patient adjustment of flow that may be inconvenient or suboptimal.

Accordingly, it would be desirable to provide improved hand-held, low flow gas dispensers that deliver a defined volume of gas to the patient at a fixed and comfortable rate of flow. Specifically, it would be beneficial to have hand-held gas dispensers that are simple to operate. It would also be desirable to have methods for treating various medical conditions with hand-held gas dispensers that improve patient compliance and provide small volumes of gas for convenient use away from the home.

### SUMMARY

Described here are hand-held, low flow gas dispensing devices for delivering therapeutic gases. The therapeutic gases may be delivered to the nasal mucosa to treat medical conditions such as headaches, allergies, asthma, and nervous disorders. The devices may be configured to control the pressure and flow rate of the delivered gas, and dispense the gas in a diffuse manner to thus improve patient comfort and compliance. With improved comfort and compliance, the devices described herein may improve treatment efficacies for the majority of patients.

The efficacy and tolerability of a nasal non-inhaled gas, e.g., CO₂, may depend upon the flow rate of the gas. A flow rate that is too low may not be effective while a flow rate that is too high may cause a more intense nasal sensation (e.g., stinging), making it less tolerable. For a device delivering a nasal gas to be useful, it generally needs to be effective while being tolerable. It is therefore beneficial to control the flow rate of the delivered gas so that it remains constant within well-defined parameters. Dispensing the gas, e.g., in a radially diffuse manner, may further minimize the nasal sensation of the gas and further improve tolerability and efficacy.

The gas dispenser devices described herein may be configured to control gas flow rates and pressures, and reduce or ameliorate unpleasant nasal sensations. The devices generally include a housing for receiving a compressed gas cylinder, a gas control assembly for controlling the flow rate and pressure of the therapeutic gas released from the cylinder, and a nosepiece configured to function as a diffusing element that reduces the nasal sensation of the dispensed gas. The reduced nasal sensation may be effected by forming the nosepiece from a material including pores having tortuous paths so that the flow of gas is diffused as it passes through the nosepiece. The nosepiece will typically reduce the stinging sensation of the dispensed gas while still providing the same pressure and flow rate of gas as if no diffusing element were used. The porous material of the nosepiece may further act as a filter for gas that flows through the nosepiece into the nasal cavity. Given that the flow of the therapeutic gas is automatically diffused by the nosepiece, the gas dispensing devices described here do not include flow adjustment features for manipulation by the patient, and are thus simple to operate.

The hand-held, low flow gas dispensers will typically include a housing having a distal end and a proximal end and a cylinder within the housing that contains a compressed therapeutic gas. A gas control assembly may be coupled to the cylinder. The gas control assembly is generally provided within the housing and proximal to the nosepiece, and typically includes a pressure regulator for adjusting and/or controlling the pressure of the gas released from the cylinder, and a gas flow outlet (e.g., a rate limiting orifice or a restrictive orifice) coupled to the pressure regulator for controlling the flow rate of the gas. As previously stated, a diffusing and filtering nosepiece may be provided at the distal end of the housing. The nosepiece may have a wall that defines a chamber, which is in fluid communication with the gas flow outlet. The wall may have a wall thickness and an internal surface area. Additionally, the wall may generally comprise a porous material having a pore size, where the porous material diffuses and filters the compressed therapeutic gas as the gas flows through the nosepiece wall.

The components of the dispenser will generally be arranged so that the pressure regulating member and the flow rate controlling member (e.g., the restrictive orifice) are provided within the housing proximal to the diffusing nosepiece. With this configuration, the pressure and flow rate of the therapeutic gas can be adjusted to a predetermined or desired flow rate prior to diffusion by the nosepiece. Given that the nosepieces described herein do not substantially restrict gas flow, the flow rate of the therapeutic gas through the nosepiece and to the patient may be substantially the same as the flow rate generated by the rate limiting orifice. By “not substantially restricting gas flow,” it is meant that when passing through the nosepiece, the flow rate of the gas is reduced by less than about 1% of the predetermined or desired flow rate. For example, if the desired or predetermined flow rate of the therapeutic gas is 0.5 SLPM (as generated by the gas control assembly, and in particular, the restrictive orifice), the flow rate of the therapeutic gas through the nosepiece may not be not restricted at all, i.e., the flow rate is the same as the desired or predetermined flow rate of 0.5 SLPM. As a further example, if there is a low degree of flow rate restriction, the 0.5 SLPM flow rate of the therapeutic gas is reduced by less than about 1% when flowing through the nosepiece.

The porous material that forms the nosepiece wall may comprise sintered ultra high molecular weight polyethylene, polypropylene, polytetrafluoroethylene (PTFE), polytetrafluoroethylene (PTFE), polyvinylidene fluoride (PVDF), ethylene vinyl acetate (EVA), high density polyethylene (HDPE), low density polyethylene (LDPE), very low density polyethylene (VLDPE), polystyrene, polycarbonate (PC) and PC/ABS blends, nylon, polyethersulfone, and combinations thereof. The inclusion of sintered ultra high molecular weight polyethylene as the porous material may be particularly beneficial. Other suitable materials that may be used to form the nosepiece include sintered metals, e.g., stainless steels, nickel, titanium, copper, aluminum, and alloys thereof.

The nosepiece of the gas dispensing devices may also have a wall thickness that optimizes radial diffusion of the gas flow. Some variations of the nosepiece will include a nosepiece wall having variable thickness. For example, the side walls of the nosepiece can be formed to be thinner than the tip of the nosepiece. The thinner walls will typically provide less resistance to the flow of gas, and thus enable more flow of gas than the thicker wall at the tip.

The compressed therapeutic gas contained within the cylinder may be any suitable therapeutic gas, e.g., carbon dioxide, nitric oxide, oxygen, helium, and combinations thereof. Some variations of the gas dispenser devices include carbon dioxide. The carbon dioxide as well as other gases may be in substantially pure form, or diluted to comprise at least 90%, at least 80%, at least 70%, at least 60%, or at least 50% of the therapeutic gas.

Some hand-held, low flow gas dispensers for intranasally delivering a therapeutic gas to a patient may comprise: a housing having a distal end and a proximal end; a cylinder within the housing and having compressed carbon dioxide contained therein; a gas control assembly coupled to the cylinder; and a diffusing and filtering nosepiece attached to the distal end of the housing, the nosepiece having a wall defining a chamber in fluid communication with the gas control assembly, the wall having a wall thickness and an internal surface area, and comprising a porous sintered ultra high molecular weight polyethylene material having a pore size, wherein the gas control assembly comprises a restrictive orifice for controlling the rate of flow of the carbon dioxide from the cylinder to the nosepiece to a desired flow rate of 0.50 SLPM, the nosepiece is constructed and arranged so as not substantially to restrict the rate of flow therethrough of the carbon dioxide and the porous sintered ultra high molecular weight polyethylene material is configured to diffuse and filter the carbon dioxide as the gas flows through the nosepiece wall.

Methods for using the hand-held, low flow gas dispensing devices to deliver a therapeutic gas, e.g., carbon dioxide, at a controlled and fixed flow rate are also described herein. In general, the method for delivering a therapeutic gas to the nasal mucosa includes inserting a nosepiece of a hand-held, low flow gas dispenser into a nasal cavity, where the nosepiece has a wall comprising a porous material having a pore size; generating a flow of therapeutic gas from a compressed gas cylinder by actuating an activation mechanism; regulating the pressure (e.g., down-regulating the pressure) and controlling the flow of the therapeutic gas released from the compressed gas cylinder using a gas flow outlet (e.g., a restrictive orifice); and diffusing the flow of the therapeutic gas as it passes through the porous material of the nosepiece wall. The step of regulating gas pressure may be accomplished using a pressure regulator having a regulator valve, a diaphragm, and a diaphragm pin assembly. The step of diffusing the flow of the therapeutic gas will generally reduce the sensation of stinging of the nasal mucosa felt by a patient. Diffusion of the therapeutic gas may be adjusted or tailored in any suitable fashion to reduce the stinging of the nasal mucosa during gas delivery. For example, the therapeutic gas may be diffused in a radial pattern, or through selective areas of the nosepiece. However, as previously stated, the nosepiece does not substantially restrict the flow rate of the gas. The method may also include filtering the flow of therapeutic gas it passes through the porous material of the nosepiece wall. Methods for treating medical conditions such as headaches (e.g., migraine headaches, cluster headaches, tension headaches, etc.); allergies (e.g., allergic rhinitis); asthma; and nervous disorders with the therapeutic gas are also described.

Alternatively, methods for delivering a therapeutic gas to the nasal mucosa may include the steps of inserting a nosepiece of a hand-held gas dispenser into a nasal cavity, the nosepiece having a wall comprising a porous material having a pore size, and the gas dispenser comprising a gas control assembly having a pressure regulator and a restrictive orifice; generating a flow of high pressure therapeutic gas from a compressed gas cylinder by actuating an activation mechanism; reducing the pressure of the therapeutic gas; controlling to a predetermined flow rate the rate of flow to the nosepiece of the reduced pressure therapeutic gas; supplying the reduced pressure therapeutic gas to the nosepiece at the predetermined flow rate; and diffusing the flow of the reduced pressure therapeutic gas as it passes through the porous material of the nosepiece wall.

Some methods for delivering a therapeutic gas to a patient's nasal mucosa comprise: inserting a nosepiece of a hand-held, low flow gas dispenser into a nasal cavity, the nosepiece having a wall comprising a porous material having a pore size, and the gas dispenser comprising, a gas control assembly having a pressure regulator and a restrictive orifice gas flow outlet; generating a flow of the therapeutic gas from a compressed gas cylinder by actuating an activation mechanism; using the pressure regulator to reduce the pressure of the generated flow of therapeutic gas; using the restrictive orifice to control to a desired flow rate the rate of flow to the nosepiece of reduced pressure therapeutic gas; supplying therapeutic gas at a reduced pressure and at the desired flow rate to the nosepiece; and diffusing the flow of the therapeutic gas as it passes through the porous material of the nosepiece wall so as to deliver the therapeutic gas to the patient's nasal mucosa substantially at the desired flow rate.

The therapeutic gases may also be used in a method of treating allergy in a patient, the method comprising the steps of inserting a nosepiece of a hand-held gas dispenser into a nasal cavity of the patient, the nosepiece having a wall comprising a porous material; and within the dispenser: generating a flow of high pressure therapeutic gas; reducing the pressure of the generated flow of high pressure therapeutic gas; controlling to a desired flow rate a rate of flow to a dispenser nosepiece of reduced pressure therapeutic gas; supplying reduced pressure therapeutic gas to the nosepiece at said desired flow rate; and diffusing the flow of the therapeutic gas as it passes through the porous material of the nosepiece wall so as to deliver the therapeutic gas to the patient's nasal mucosa substantially at said desired flow rate. The desired flow rate may range from between 0.20 and 1.00 standard liters per minute (SLPM), between 0.35 to 0.65 SLPM, or between 0.40 and 0.60 SLPM. The therapeutic gas for use in a method of treating allergy in a patient may comprise: inserting a nosepiece of a hand-held gas dispenser into a nasal cavity of the patient, the nosepiece having a wall comprising a porous material; and within the dispenser: generating a flow of high pressure therapeutic gas; reducing the pressure of the generated flow of high pressure therapeutic gas; controlling to a desired flow rate a rate of flow to a dispenser nosepiece of reduced pressure therapeutic gas; supplying reduced pressure therapeutic gas to the nosepiece at said desired flow rate; and diffusing the flow of the therapeutic gas as it passes through the porous material of the nosepiece wall so as to deliver the therapeutic gas to the patient's nasal mucosa substantially at said desired flow rate. Here the step of regulating gas pressure (e.g., reducing gas pressure) may be accomplished using a pressure regulator having a regulator valve, a diaphragm, and a diaphragm pin assembly. The step of diffusing the flow of the therapeutic gas will generally reduce the sensation of stinging of the nasal mucosa felt by a patient. Diffusion of the therapeutic gas may be adjusted or tailored in any suitable fashion to reduce the stinging of the nasal mucosa during gas delivery. For example, the therapeutic gas may be diffused in a radial pattern, or through selective areas of the nosepiece. However, as previously stated, the nosepiece does not substantially restrict the flow rate of the gas. The method may also include filtering the flow of therapeutic gas it passes through the porous material of the nosepiece wall.

Methods of assembling a hand-held, low flow gas dispenser for intranasally delivering a therapeutic gas to a patient via a diffusing and filtering nosepiece assembled to a dispenser gas outlet are also described herein. In the methods, the dispenser may generally include a gas control assembly including a pressure regulator for reducing the pressure of gas supplied thereto and a restrictive orifice for controlling the rate of flow of reduced pressure gas supplied thereto by the pressure regulator. Here the method may further include the sequential steps of: adjusting the pressure regulator to provide gas to the dispenser outlet, when the nosepiece is not assembled thereto, at a desired delivery pressure and flow rate; and assembling the nosepiece to the dispenser gas outlet so as to enable gas to be intranasally delivered to the patient substantially at the desired delivery pressure and flow rate via the assembled nosepiece. Thus, a method of assembling a hand-held, low flow gas dispenser for intranasally delivering a therapeutic gas to a patient via a diffusing and filtering nosepiece assembled to a dispenser gas outlet, the dispenser having a gas control assembly including a pressure regulator for reducing the pressure of gas supplied thereto and a restrictive orifice for controlling the rate of flow of reduced pressure gas supplied thereto by the pressure regulator, may comprise the sequential steps of: adjusting the pressure regulator to provide gas to the dispenser outlet, when the nosepiece is not assembled thereto, at a desired delivery pressure and flow rate; and assembling the nosepiece to the dispenser gas outlet so as to enable gas to be intranasally delivered to the patient substantially at the desired delivery pressure and flow rate via the assembled nosepiece. Assembly according to these methods may be useful given that the manner of assembly allows the gas flow rate to be controlled to a desired flow rate, and because the gas at the desired flow rate is automatically diffused by the nosepiece, the gas dispensing devices described herein do not include or require flow adjustment features for manipulation by the patient, and are thus simple to operate.

### BRIEF DESCRIPTION OF THE DRAWINGS

FIGS. 1A-1C depict various views of a hand-held, low flow gas dispensing device according to one variation. FIG. 1A shows a perspective view of the gas dispensing device, FIG. 1B is line drawing showing a side view of the gas dispensing device, and FIG. 1C is a further line drawing showing a front view of the gas dispensing device.

FIGS. 2A-2B show expanded views of an exemplary nosepiece of a gas dispensing device. FIG. 2A depicts a side view of the nosepiece. FIG. 2B depicts a cross-sectional view of the nosepiece along line A-A as shown in FIG. 2B.

FIG. 3 shows a microscopic view of an exemplary nosepiece material.

FIG. 4 is a graph showing comparative flow data between a gas dispenser using an exemplary diffusing nosepiece and a gas dispenser lacking the diffusing nosepiece.

FIG. 5 depicts an expanded cross-sectional view of a gas control assembly according to one variation.

FIGS. 6A-6B depict exemplary configurations of the gas control assembly of FIG. 5 within the gas dispenser housing.

FIG. 7 is a graph showing that the gas dispensers described herein are capable of maintaining a relatively constant gas flow rate with temperature changes.

FIG. 8 depicts an expanded, cross-sectional view of a nosepiece and rate limiting orifice (gas flow outlet) according to one variation.

FIG. 9 shows an expanded, cross-sectional view of a pierce pin assembly and stem valve according to one variation.

FIG. 10 depicts a cross-sectional view of an exemplary gas control assembly.

FIG. 11 shows a gas dispenser actuator according to one variation.

FIG. 12 depicts a cross-sectional view of an exemplary nosepiece according to another variation.

### DETAILED DESCRIPTION

Described here are hand-held, low flow gas dispensing devices for delivering a therapeutic gas to the nasal mucosa. The devices may be configured to control the pressure and flow rate of the delivered gas, and diffuse gas flow to thus improve patient comfort and compliance. The gas flow rate will typically be controlled to a predetermined or desired rate of flow that it is not too low (and therefore ineffective in treating symptoms of a medical condition, e.g., allergic rhinitis), and not too high (and therefore intolerable). The flow rate of the therapeutic gas is not substantially restricted as it diffuses through the nosepiece of the dispensing devices.

The devices generally include a housing for receiving a compressed gas cylinder and a gas control assembly. The gas control assembly is generally configured to include a pressure regulator for controlling the pressure of the gas released from the cylinder and a restrictive orifice for controlling to a desired or predetermined flow rate the flow rate of the gas. The gas dispensing devices may also include a nosepiece configured to function as a diffusing and/or filtering element that reduces the nasal sensation of the dispensed gas. Multiple dispenses of the therapeutic gas can be delivered from a single compressed gas cylinder, e.g., from 10 to 80 or more. In some variations, the number of therapeutic gas dispenses ranges from 10 to 60 or 10 to 40.

As previously stated and further described below, the reduced nasal sensation may be effected by forming the nosepiece from a porous material including pores having tortuous paths so that the flow of gas is diffused as it passes through the nosepiece. The nosepiece will typically reduce the stinging sensation of the dispensed gas while still providing the same pressure and flow rate of gas as if no diffusing element were used. The porous material of the nosepiece may further act as a filter for gas that flows through the nosepiece into the nasal cavity. Given that the flow of the therapeutic gas is automatically diffused by the nosepiece, the gas dispensing devices described here do not include flow adjustment features for manipulation by the patient, and are thus simple to operate. In another variation, the nosepiece is formed from a material that can be laser drilled to create holes or openings therethrough. Here the hole size and geometry can be set to preselected values, and hole placement in the nosepiece can be provided in preselected locations or in a particular pattern.

Hand-Held, Low Flow Devices

The hand-held, low flow gas dispensers generally include a housing having a distal end and a proximal end, a cylinder within the housing that contains a compressed therapeutic gas, a gas control assembly coupled to the cylinder for controlling the pressure and flow of the dispensed gas, and a diffusing and filtering nosepiece at the distal end of the housing configured to gently and effectively deliver gas to a nostril. In general, the gas dispenser includes a compressed gas cylinder containing between 4 to 16, or between 7 to 16 grams of liquid and gaseous carbon dioxide, a piercing mechanism to pierce the sealed gas cylinder and allow the flow of compressed gas into the controlling/regulating portion of the device (gas control assembly), an on/off valve that is manually operated by the user to commence and cease the flow of gas. The gas control assembly may include a pressure regulating element to down-regulate the gas cylinder pressure to a comfortable range of pressure suitable to intranasal administration, and a restrictive orifice (gas flow outlet) to control the rate of flow of the gas.

The gas dispensers described herein may provide controlled flow rates across a range of temperatures that the device could be expected to encounter (i.e., 10° C. to 40° C.). The optimal flow rate may be considered to be between 0.20 and 1.00 standard liters per minute (SLPM), between 0.35 to 0.65 SLPM, or between 0.40 and 0.60 SLPM. A “dose” may be defined as a predefined volume or mass of gas delivered to the patient. This may be achieved by controlling both the flow rate of the gas and the total duration of the time of delivery. Exemplary doses may include dispensing the therapeutic gas at 0.50 SLPM for between about 5 to about 90 seconds, between about 5 to about 20 seconds, or between about 5 to about 10 seconds.

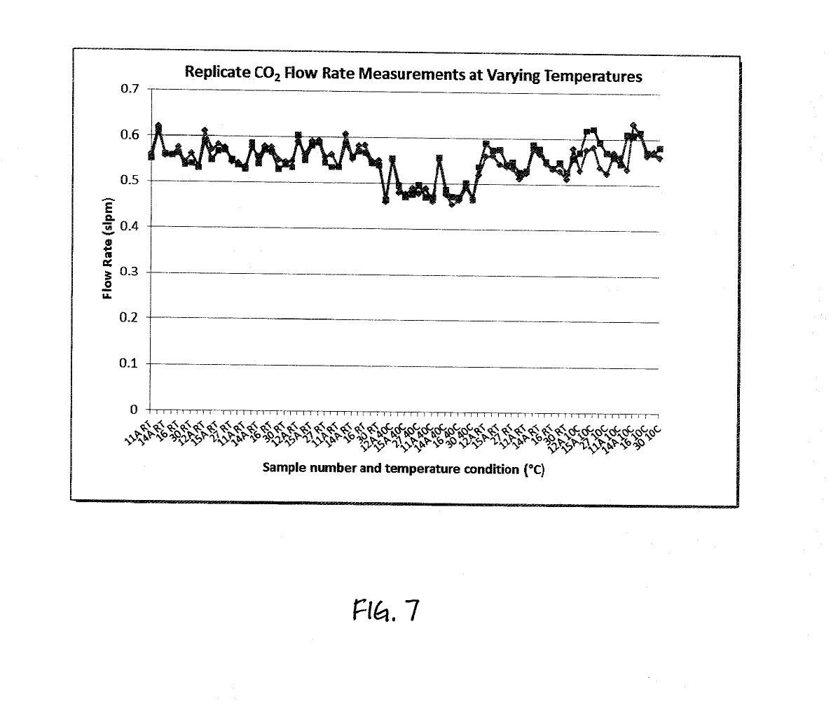

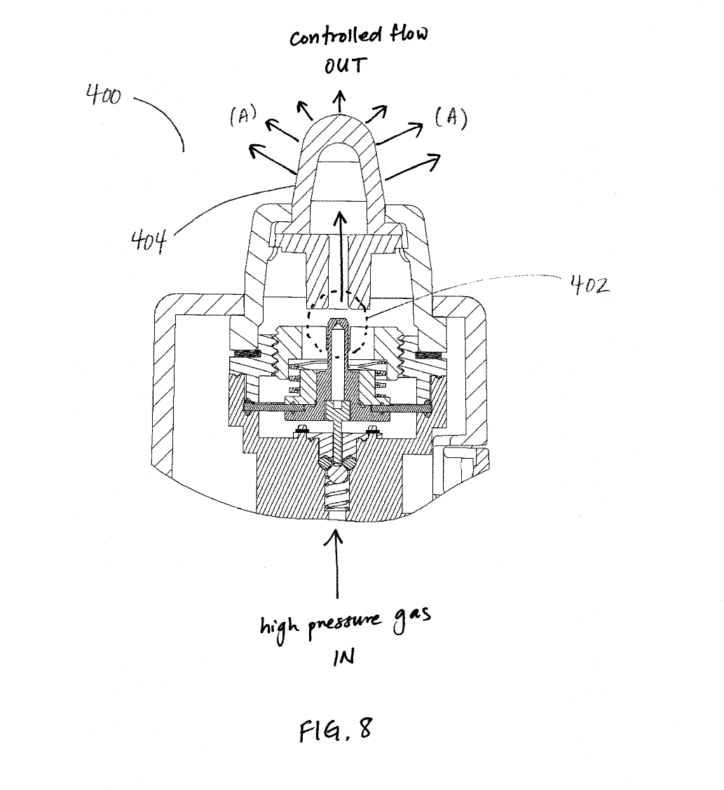

The flow rate of the gas may be controlled by down-regulating the gas pressure from a typical cylinder pressure of 850 psig (pounds per square inch gauge) (approximately 58 atm) to approximately 14.7 psig (pounds per square inch gauge) (1 atm), and dispensing the down-regulated gas through a rate controlling orifice (gas flow outlet) of a precise size. This approach may have the advantage of compensating for moderate temperature changes that the hand-held device will encounter. For example, the nominal gas pressure of carbon dioxide at 22° C. of 58 atm may increase to nearly 82 atm at 40° C. Conversely, the gas cylinder pressure may drop to approximately 44 atm at 10° C. Because the gas pressure is first down-regulated to approximately 1 atm, these temperature excursions may not significantly change the flow rate of the dispensed gas, as shown in FIG. 7.

Diffusing and Filtering Nosepiece

The substantially non-obstructing/non-restrictive nosepiece of the gas dispensing devices described herein will generally be fixedly or removably (e.g., by crimping, welding, friction-fit, snap-fit, or screw type mechanisms, etc.) attached to the distal end of the device housing. The nosepiece is substantially non-obstructing/non-restrictive because it does not substantially alter the flow rate of the gas flowing through it. In general, the flow rate of the therapeutic gas is reduced by less than 1% of the desired or predetermined flow rate generated by the restrictive orifice when the gas flows through the nosepiece. The nosepiece may have any suitable size, shape, and geometry. For example, the nosepiece may be rounded and tapered toward its tip. In some variations, the height of the nosepiece may range from about 1 cm to about 2 cm. The height may be about 1.2 cm in one variation. The width of the nosepiece at its base may range from about 0.5 cm to about 1 cm. In some instances a width of about 0.8 cm or about 0.9 cm may be useful.

The nosepiece will generally comprise a wall that defines an outer surface and an inner surface. The wall may comprise a porous material including, but not limited to, sintered ultra high molecular weight polyethylene, polypropylene, polytetrafluoroethylene (PTFE), polytetrafluoroethylene (PTFE), polyvinylidene fluoride (PVDF), ethylene vinyl acetate (EVA), high density polyethylene (HDPE), low density polyethylene (LDPE), very low density polyethylene (VLDPE), polystyrene, polycarbonate (PC) and PC/ABS blends, nylon, polyethersulfone, and combinations and thereof. In one variation, the porous material is sintered ultra high molecular weight polyethylene. The sintered porous plastic nosepiece may contain an open cell structure continuously throughout such that gas will emit from all surfaces of the component. The nosepiece material will generally be hydrophobic, which is a typical property of most thermoplastics. The hydrophobicity, if required, can be enhanced with various coatings or surface treatments. One benefit of a hydrophobic porous plastic nosepiece may be the component's ability to repel adhesion of nasal mucus. This is especially important where the medical condition being treated (e.g., allergic rhinitis) will likely produce nasal congestion. It may be a further benefit that a hydrophobic component will be easier to clean and will have less tendency to become clogged than a hydrophilic structure. Other suitable materials that may be used to form the nosepiece include sintered metals, e.g., stainless steels, nickel, titanium, copper, aluminum, and alloys thereof.

The diffusion (and filtering) properties of the nosepiece may be manipulated by adjusting one or more such factors as the surface area of the inner wall surface, wall thickness, porosity of the material used, and pore size. For example, diffusion of the therapeutic gas may be enhanced if the gas contacts and flows through a larger surface area of the inner wall surface. However, as previously stated, the rate of gas flow through the nosepiece is substantially the same as its flow rate from the rate controlling orifice (i.e., the flow rate of the gas is not substantially restricted as it travels through the material of the nosepiece).

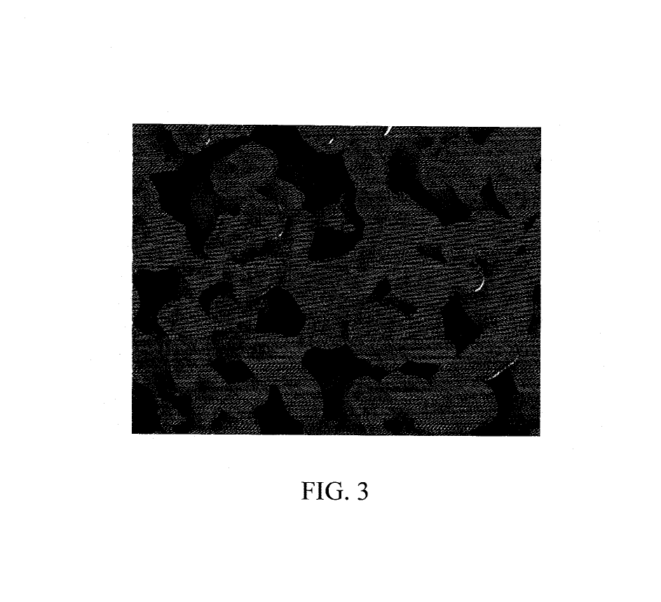

The pore size that may be useful in diffusing and filtering the therapeutic gas flowing through the nosepiece ranges between about 10 microns and about 100 microns, or between about 15 microns and about 50 microns, or between about 20 microns and about 28 microns. In some variations, the porous material has a pore size of about 24 microns. An exemplary photograph of the porous material (here sintered ultra high molecular weight polyethylene) is shown in FIG. 3. The tortuous nature of the pores in the material of the nosepiece may also confer diffusion and filtering benefits. An exemplary way of making the porous material of the nosepiece is described in Example 1. Although the nosepiece can be homogeneously formed with pores throughout, it can also be made to have pores heterogeneously distributed in the nosepiece, or formed in discreet areas of the nosepiece to better control the direction of diffusion. For example, the pores may be distributed so that a substantial amount of the pores are located in side walls of the nosepiece (instead of the tip) to effect radial diffusion of the therapeutic gas (as opposed to concentrating diffusion through the tip of the nosepiece).

The wall thickness of the nosepiece may also be adjusted or varied to optimize gas diffusion, e.g., radial diffusion or diffusion through desired areas. For example, a thinner wall may be used in some areas to provide less resistance of the flow of gas while, conversely, thicker walls may be used in other areas to provide greater flow resistance and, hence, less flow of gas. In some variations, the side walls of the nosepiece are substantially thinner than the end or tip of the nosepiece. In one variation, the wall thickness ranges from about 0.10 to about 0.35 cm or from about 0.15 cm to about 0.25 cm. In another variation, the wall thickness is about 0.17 cm. In yet further variations, nosepiece walls having variable thickness are employed.

A hand-held, low flow gas dispenser having a nosepiece where gas flow is radial and diffuse may be particularly beneficial. Clinical studies conducted by the assignee of the instant patent application evaluated whether carbon dioxide delivered via a nosepiece that diffused (e.g., diffused in a radial fashion) its flow was better tolerated (e.g., less stinging was sensed) than one which did not (i.e., allowed the carbon dioxide to flow directly through into the nasal cavity). The data showed that the nosepiece that generated a diffuse flow to the nasal mucosa caused less nasal stinging than the one having a direct flow of carbon dioxide.

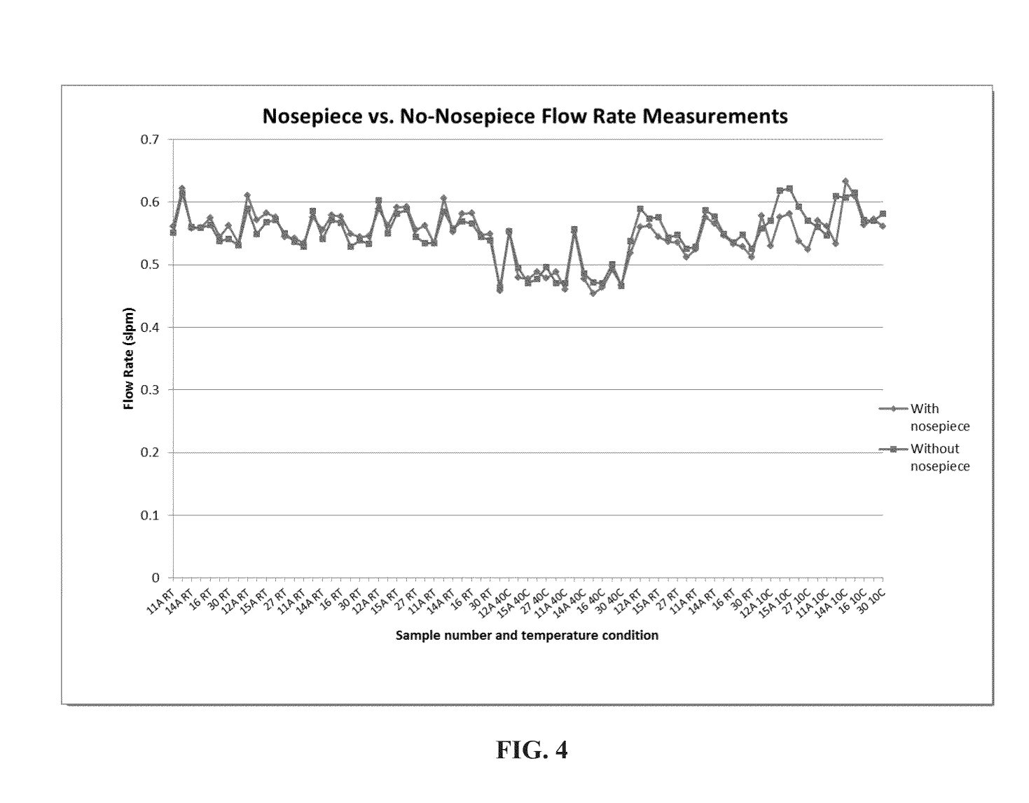

Furthermore, experiments performed using the diffusing and filtering nosepiece described herein have demonstrated that the nosepiece does not obstruct gas flow or effect gas pressure. Referring to FIG. 4, the graph shown therein shows that the filtering, diffusing nosepiece does not restrict gas flow. The graph reflects comparative data between hand-held gas dispensers having a diffusing, filtering nosepiece and those without the nosepiece at three (3) different temperature conditions: room temperature (RT), 40° C., and 10° C. Since the data is nearly overlapping, there is no substantial restriction of flow introduced by the use of the nosepiece. Again, by “no substantial restriction,” it is meant that that when passing through the nosepiece, the flow rate of the gas is reduced by less than about 1% of the predetermined or desired flow rate.

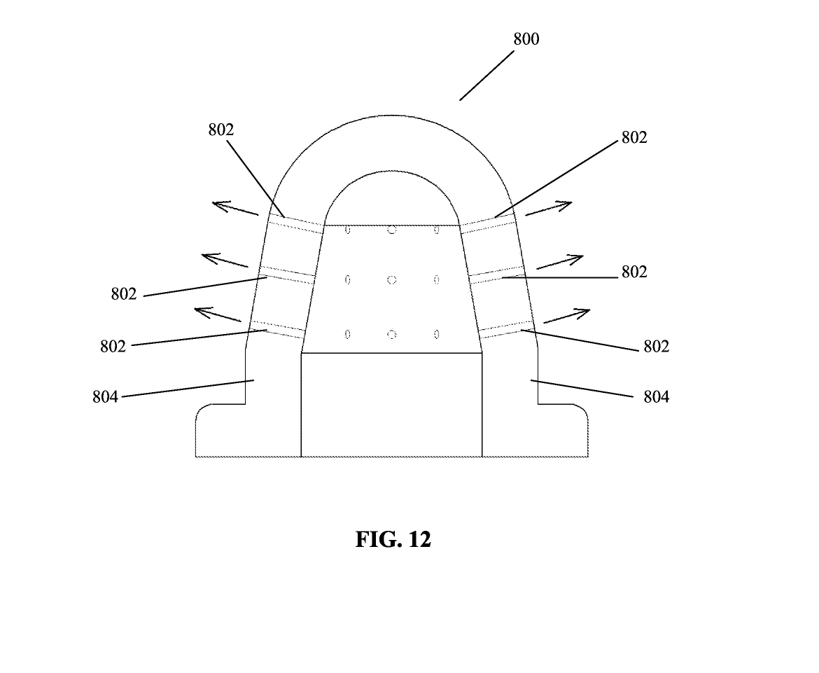

Alternatively, the nosepiece can be formed from a broad range of materials and laser drilled with holes of any suitable size and geometry. The holes may also be laser drilled in any suitable distribution or pattern, so long as the distribution or pattern does not substantially restrict the flow of therapeutic gas therethrough. Forming may be achieved through plastic injection molding or machining, for example, with materials including, but not limited to, rigid thermoplastics such as ABS, polycarbonate, nylon, polyester, liquid crystal polymers, PEEK, polyamide-imide, polyetherimide, polyethersulfone, POM, polysulfone, PVC, polystyrene, and acrylic. In general, rigid thermoplastics can readily be laser drilled with holes having a size ranging from about 50 microns to about 100 microns. However, hole sizes of less than 50 microns or greater than 100 microns may also be made. Further, this drilling can be performed at high volume commercial manufacturing scales with great accuracy and high speed, making the process economically feasible and yielding a high quality and repeatable component. Referring to the cross-sectional view of FIG. 12, an exemplary nosepiece (800) has a plurality of holes (802) laser drilled through the nosepiece side walls (804) for the passage and diffusion of a therapeutic gas (806) from the interior of the nosepiece (800) in the direction of the arrows. Although three holes in each side wall are shown in FIG. 12, any suitable number and configuration of holes may be machined.

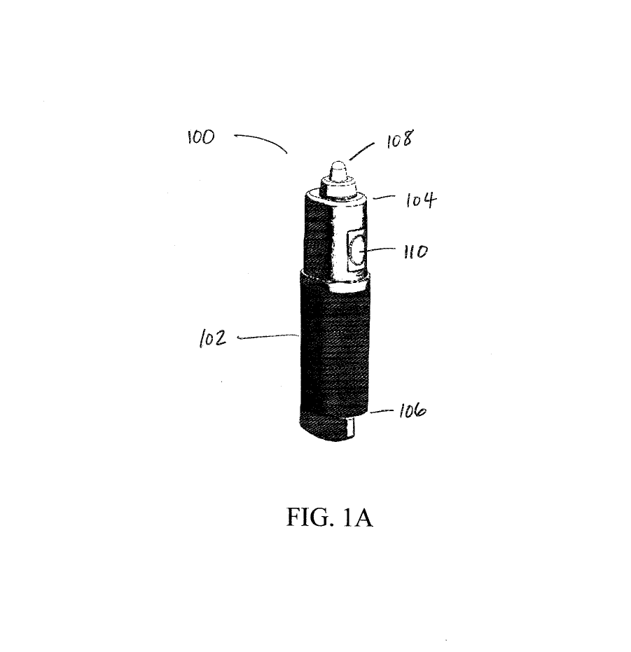

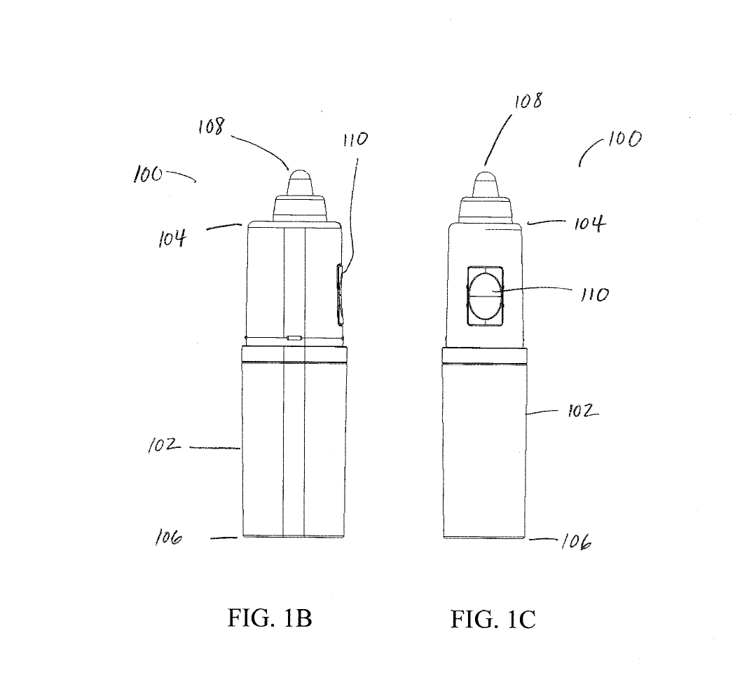

Referring to FIGS. 1A-1C, an exemplary hand-held, low flow gas dispenser is shown. Gas dispenser (100) includes a housing (102) having a distal end (104) and a proximal end (106), and a nosepiece (108) at the distal end (104) of the housing (102). The housing (102) may be about 12.5 cm in length, or range from between about 7 cm to about 13 cm in length. A removable cover (not shown) may be provided over the nosepiece (108). Although a push button (110) (e.g., to turn the dispenser on and off) is shown for actuating release of a therapeutic gas from the compressed gas cylinder residing within the housing (102), other modes of actuation may be contemplated.

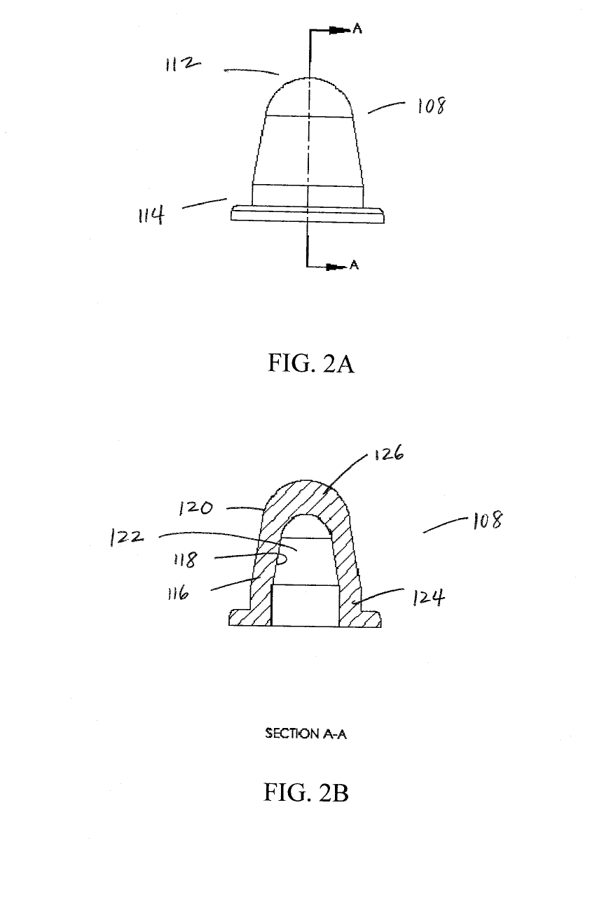

FIG. 2 shows an expanded view of the nosepiece (108) in FIG. 1. Here the nosepiece has a distal end (112) and a proximal end (114). The distal end (112) is rounded and slightly tapered as it progresses from the proximal end (114) to the distal (114). However, as previously stated, the nosepiece may have any suitable configuration. A cross-sectional view of the nosepiece (108) of FIG. 2A is shown in FIG. 2B, as taken along line A-A. Here a wall (116) of the nosepiece is shown having an inner surface (118) and an outer surface (120). The wall (116) of the nosepiece defines a chamber (122) that is in fluid communication with a gas flow outlet of the device. Again, diffusion of the therapeutic gas may be enhanced if the gas contacts and flows through a larger surface area of the inner wall surface. The wall at the proximal end of the nosepiece (124) is less thick than the wall at the distal end of the nosepiece (126). The flow rate of a therapeutic gas is not substantially restricted as it passes through nosepiece (108). Other suitable configurations of the nosepiece may also be contemplated.

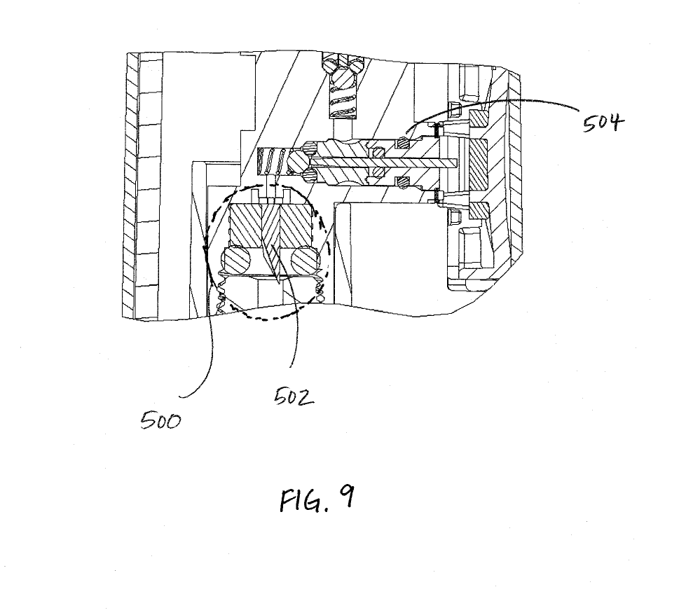

In some variations, and as shown in FIG. 8, a gas dispenser (400) may expel low-pressure gas through a rate controlling orifice (gas flow outlet) (402), and through a porous plastic diffusing and filtering nosepiece (404) in the direction of arrows (A). But as previously stated the nosepiece may also be made from a sintered metal. Furthermore, and as stated earlier, higher gas flow rates can result in improved patient efficacy but also commonly result in increased nasal sensations such as stinging and burning. It may be an advantage of the devices described here that they provide a diffuse radial pattern of gas delivery in the nostril that has been found to reduce unwanted nasal sensations, particularly nasal stinging. Again, the reduced stinging sensation may be effected by forming the nosepiece from a material including pores having tortuous paths so that the flow of gas is radially diffused as it passes through the nosepiece. The nosepiece will typically reduce the stinging sensation of the dispensed gas while still providing the same pressure and flow rate of gas as if no diffusing element were used. The porous material of the nosepiece may further act as a filter for gas that flows through the nosepiece into the nasal cavity.

Gas Control Assembly

The gas control assembly included in the hand-held, low flow gas dispenser devices described herein generally controls the pressure and flow rate of the therapeutic gas released from the cylinder. Some variations of the device comprise a gas control assembly having elements arranged into a single, compact, and low-cost design that adjusts, e.g., down-regulates the pressure of a source gas, provides for the activation and cessation of the delivery of the gas via an on/off valve, and precisely controls the flow rate of the gas, all in a single unit.

The gas control assembly is generally designed for coupling to a high pressure gas cylinder, such as a miniature, disposable, pressurized carbon dioxide cylinder, activate the flow of gas from the cylinder via a pierce pin and sealing member (o-ring), and provide delivery of the gas to the activation means (on/off valve). The component, in a single, low-cost assembly, may thus provide a means to access the source gas cylinder, selectively activate or cease the flow of the gas, control the delivery of the gas from a high source pressure (e.g., 850 psig nominal pressure) to a low delivery pressure (e.g., 14.7 psig), and control the flow rate of the gas at a desired level (e.g., 0.50 SLPM). The gas control assembly may be configured to control or adjust the flow rate of the therapeutic gas to between about 0.30 SLPM to about 0.70 SLPM. Some variations of the gas control assembly may control or adjust the flow rate of the therapeutic gas to between about 0.40 SLPM to about 0.60 SLPM.

The gas cylinder may be a conventional-type miniature cylinder that contains a therapeutic gas, e.g., between 4 grams and 16 grams, or between 7 grams and 16 grams of pressurized carbon dioxide. The internal pressure at room temperature (21° C.) and a liquid fill volume of approximately 75% is approximately 850 psi (58 atm). The internal pressure of the gas will increase or decrease with higher or lower temperature, respectively, and will vary from between about 1200 psi (82 atm) at 40° C. and 650 psi (44 atm) at 10° C. The cylinder may be constructed of mild steel and may be capable of withstanding pressures in excess of 50 MPa (490 atm). The cylinder may contain a sealing cap or metal septum that is pierceable and the cylinder may have a threaded or non-threaded neck. Such gas cylinders are commercially available from companies such as iSi GmbH, Liss, Leland Ltd., Nippon Tansan Gas Co. Ltd., etc.

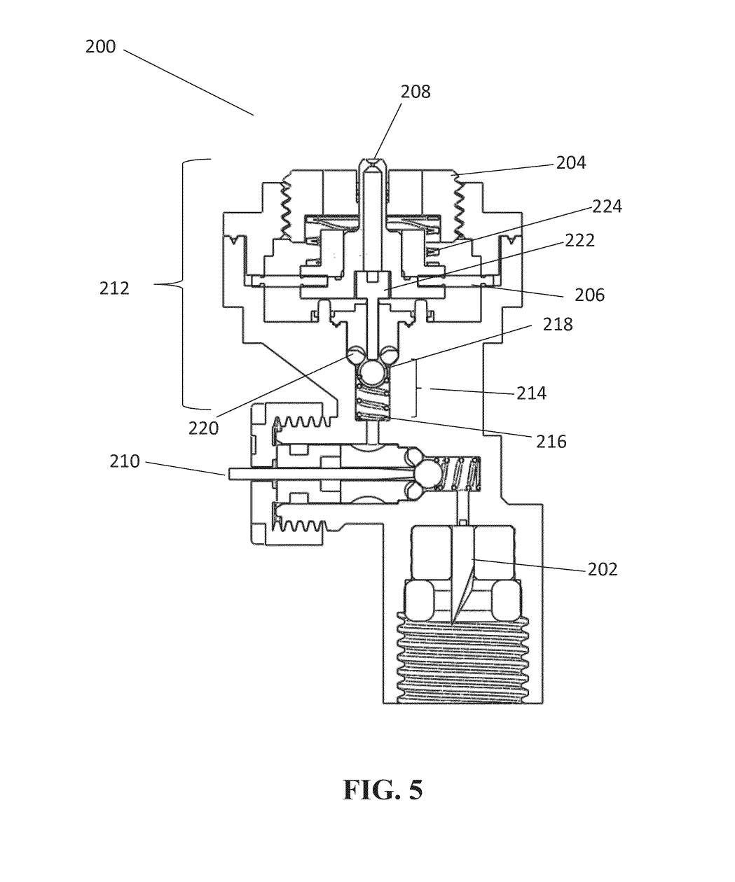

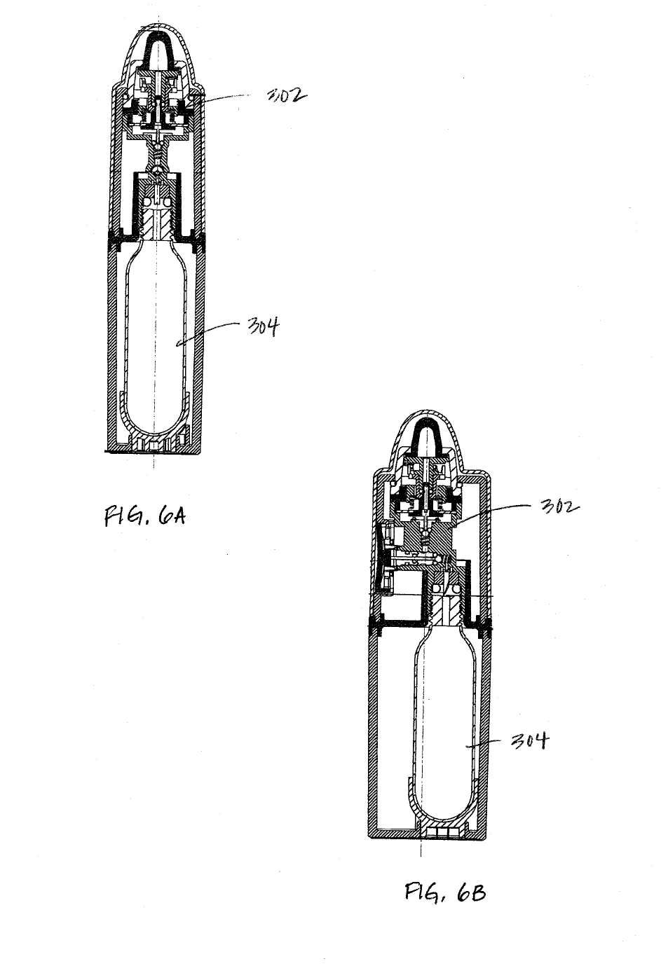

In some variations, the gas control assembly may be configured as shown in FIG. 5. In the figure, the gas control assembly (200) is provided with a pierce assembly (202) that provides access of the therapeutic gas from the compressed gas cylinder (not shown). The combination of a flow rate adjustment screw (204), pressure regulating diaphragm (206), and limiting orifice (gas flow outlet) (208) down-regulates the pressure of the source gas and precisely controls the flow of gas, all in a single unit. A stem valve assembly (210), which can be actuated, e.g., by a push button, may also be included to activate the flow of gas from the cylinder. The gas control assembly (302) may be provided in the gas dispensers either in-line with the compressed gas cylinder (304) (FIG. 6A) or offset with the compressed gas cylinder (304) (FIG. 6B and FIG. 5).

As shown in more detail in FIG. 9, the piercing mechanism (500) may be comprised of a penetrating pin or needle (502) that pierces the gas cylinder cap and allows flow of the gas in such a manner that the rate of flow is not restricted. Such pierce pin arrangements are well understood and widely used in the industry and may comprise any number of suitable pin arrangements, with or without a subsequent filtering element such as a sintered frit. Hollow steel piercing pins are a commonly used example. Here the piercing pin may penetrate the cylinder cap and remain in place in the cap while allowing gas to flow through the pin. It should be understood that this piercing mechanism arrangement is not intended to regulate gas flow. Further, it should be understood that the gas dispensers described here do not use the pierce pin to shut-off the flow of gas. Rather, a stem valve mechanism (504) is generally used for that purpose. For example, the dispensed gas flows through the piercing mechanism (500) into the stem valve mechanism (504) that either permits the flow of gas to continue to the pressure regulating element (i.e., in the open position) or completely shuts-off the flow of gas (i.e., the closed position). The stem valve (504) may be manually operated by the user to commence or cease the flow of gas. Some variations of the stem valve mechanism employ a ball-type stem valve where a normally closed ball seals against an o-ring, preventing the flow of gas. Here the ball may be spring-loaded, which causes it to normally be closed (i.e., pressed against the o-ring to form a gas-tight seal). To commence the flow of gas, the user actuates, e.g., by pushing a button that, in turn, pushes a pin against the ball, dislodging it from the o-ring and allowing gas to flow through the o-ring and downstream in the device. The stem valve mechanism is typically of simple and compact design, using very small components to reduce the entrained volume of gas. In so doing, the pressures exerted by the gas against the ball and the overall mechanism may be minimized. The ball diameter, for example, may be about 0.2 cm (about 0.079″). With a nominal gas pressure of 850 psig exerted against the ball, the resulting force applied to the ball is 1.3 pounds. Consequently, the activation force needed to force the ball away from the o-ring is 1.3 pounds plus the force applied by the returning spring. The gas dispenser devices described here employ a spring with a force of about 2 pounds. As a result, the user must apply a manual force of about 3.3 pounds (1.5 kgf) to commence the flow of gas. Releasing the on/off button by the user causes the ball to return to its normally closed position as a result of both the spring force applied against the ball and the gas pressure exerted on the ball's surface. This stem valve mechanism, like the pierce mechanism, does not restrict the flow or pressure of the gas.

When the stem valve mechanism is open, gas may flow to the gas control assembly where the gas pressure is down-regulated from the nominal gas cylinder pressure of about 850 psig, or 58 atm, to approximately 14.7 psig, or about 1 atm. The gas control assembly comprises a single-stage, diaphragm-type pressure regulator that controls the pressure of the outgoing gas with considerable precision. The output pressure from the regulator can be pre-set via an adjustment screw during manufacture to any desirable pressure. In the gas dispenser devices described herein, this element (gas control assembly) may be highly miniaturized and compact, having a diameter of approximately 22 mm and an overall height of about 15 mm.

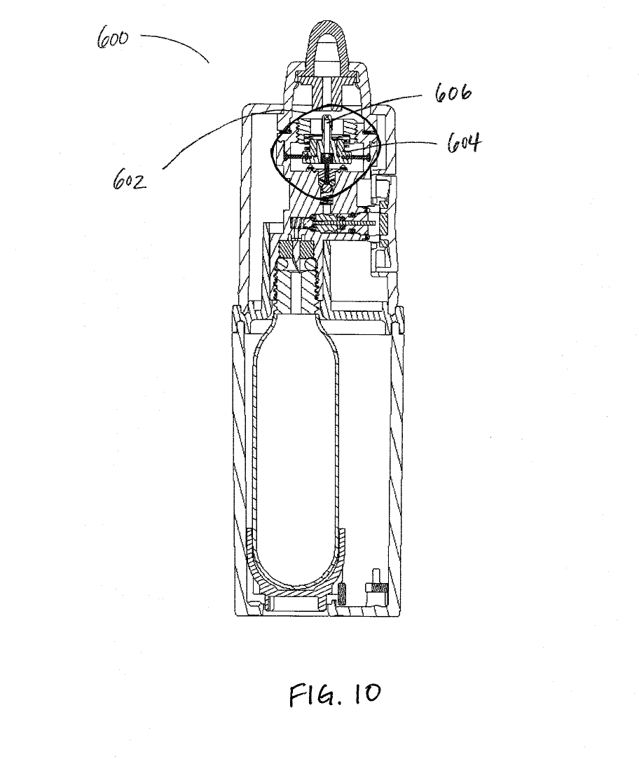

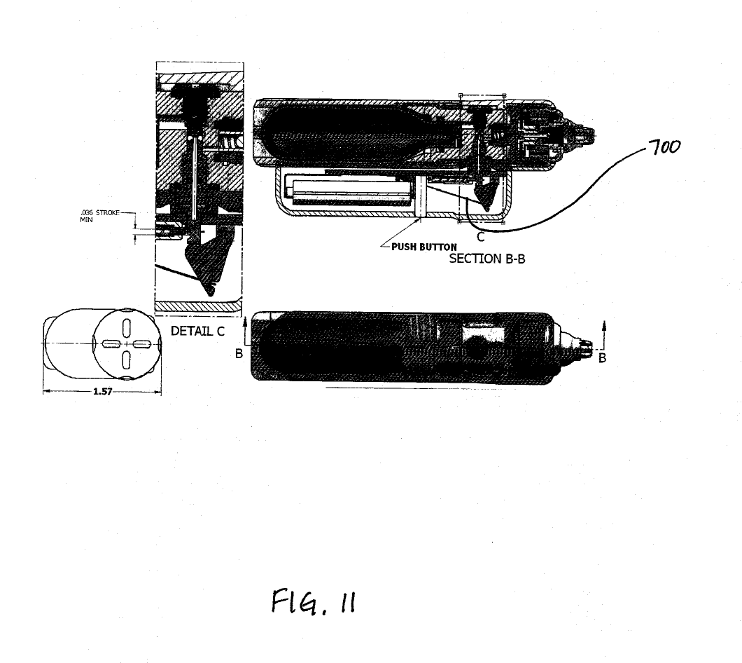

Referring to FIG. 10, the flow and pressure control aspect of the hand-held devices (600) may be due to the inclusion of a gas control assembly (602). The gas control assembly (602) may be comprised of two components—a pressure regulator (604) and a rate controlling orifice (gas flow outlet) (606). These two components may generally work in unison to obtain the desired (or a predetermined) gas flow rate—a gas at a given pressure, passing through a given orifice, will typically flow at a controlled rate.

An exemplary pressure regulator (as shown in FIG. 5) may be comprised of three components, working in conjunction to regulate the output pressure. The first component may be a pressure regulator (212) having a regulator valve (214). The regulator valve (214) may be comprised of a small spring (216), ball (218), and sealing o-ring (220). The functionality of this valve may be similar to the on/off valve in the device; when the ball (218) is in contact with the sealing o-ring (220), no gas is allowed to pass from the inlet side to the pressure chamber of the regulator. The valve mechanism (214) may be mechanically linked to the diaphragm (206) via the diaphragm pin (222).

The second component may be the diaphragm (206) and diaphragm pin assembly. The diaphragm may be comprised of a soft elastomeric bellows, e.g., formed from a silicone material having a Shore A hardness ranging from about 40 to 90 or from about 50 to 80. The diaphragm (206) may be used to develop the chamber area that will be pressurized to the desired pressure, as well as allow unhindered axial movement of the diaphragm pin (222). The diaphragm pin (222) may be used to translate this axial motion of the diaphragm (206) to the regulator valve (214).

The third component may be the regulating spring (224) and adjustment screw (204). This spring (224) generally applies a force to the diaphragm (206), to counter the opposing force from the gas pressure inside the regulator chamber. The force exerted by the spring (224) may be adjusted by the adjustment screw (204). Subsequently, the higher the load exerted by the spring (224), the higher the pressure required in the regulator chamber to counter this force and close the valve (214).

In some variations, these three components work in unison as follows. When the device is not activated (on/off valve is closed), the force developed by the regulating spring (224) pushes on the diaphragm (206), which in turn pushes on the ball (218) via the diaphragm pin (222), subsequently keeping the gas flow path open. Once the device is activated, gas will flow past the regulator valve (214) into the diaphragm chamber. As the diaphragm chamber pressurizes, it will begin to exert a countering force against the spring (224) thus allowing the diaphragm pin (222) to move away from the regulator valve (214), which in turn allows the valve to close. Since the length of travel required to close the valve is constant, the amount of force exerted by the spring at the given set point will be also constant. This in turn means that the pressure required to close the valve (214) will be constant as well. Thus, the regulator pressure can be very accurately controlled by the amount of preload applied to the spring (224) via the adjustment screw (204).

As previously stated, the gas control assembly may include a rate limiting orifice (gas flow outlet). The rate limiting orifice may be used to control the flow rate. The rate limiting orifice may be configured to have a diameter that ranges from about 0.015 cm (0.006 in) to about 0.025 cm (0.010 in). In some variations, the rate limiting orifice has a diameter of about 0.020 cm (0.008 in). As the pressure in the regulator is increased, the flow of gas past the rate limiting orifice also increases. Conversely, if the pressure inside the regulator is decreased, the flow of gas past the rate limiting orifice will decrease. In view of these principles, a well-controlled flow rate can be established by adjusting the regulator pressure, via, e.g., an adjustment screw.

The therapeutic gas dispensed by the hand-held devices described here may be carbon dioxide, nitric oxide, oxygen, helium, and combinations thereof. The therapeutic gas may comprise essentially pure carbon dioxide or other pure therapeutic gas. By “essentially pure,” it is meant that the carbon dioxide, or other therapeutic gas, is free from the significant presence of other gases, i.e., the total volume of gas will comprise at least 50% carbon dioxide, preferably at least 70% carbon dioxide, and more preferably 95% or greater.

In other variations, physiologically or biologically active components (such as drugs), saline, etc., may be delivered along with the therapeutic gas from the dispensing devices. In some variations, a combination of carbon dioxide and saline are dispensed to the nasal mucosa.

In other variations, however, the carbon dioxide, or other therapeutic gas, may be present in a carrier that would have a significant presence, i.e., the total volume of carbon dioxide will comprise at least 6% carbon dioxide, preferably at least 30% carbon dioxide, and more preferably 49%. The carrier may be inert or biologically active. Exemplary inert carrier gases include nitrogen, air, oxygen, halogenated hydrocarbons, and the like.

Alternative variations of the gas dispensing devices may incorporate a 555 timer IC and a beeper (such as a piezo element) that commences a countdown from the time the on/off button is pushed and audibly beeps after a predetermined duration (e.g., 10 or 20 seconds). The audible beep may notify the user to cease dispensing. The timer and beeper may be integrated onto a single, very small PC board that contains a coin cell battery. An onboard timer would be a convenience to the user so that they do not have to reference a watch or a clock to monitor the dispense duration.

A further variation of the gas dispensing devices is to include an actuation means that automatically turns-on and shuts-off the flow of gas at the end of the dispense duration. In this variation, a mechanism may be added to the unit such that the entire dispensing sequence is initiated with a single push of the on/off button by the user. Following this button push, the unit automatically dispenses gas for the prescribed duration and then automatically shuts-off. One means of achieving this is with the use of a nitinol wire actuator (700) such as that shown below in FIG. 11.

Methods

Methods for delivering a therapeutic gas to the nasal mucosa are also described herein. In general, the method includes the steps of inserting a nosepiece of a hand-held, low flow gas dispenser into a nasal cavity, the nosepiece having a wall comprising a porous material having a pore size; generating a flow of a therapeutic gas from a compressed gas cylinder by actuating an activation mechanism; and diffusing the flow of therapeutic gas as it passes through the porous material of the nosepiece wall. The gas dispenser generally comprises a gas control assembly having a pressure regulator and a gas flow outlet. Pressure of the therapeutic gas released from the cylinder may be controlled (e.g., adjusted to down-regulate the pressure) by the pressure regulator. Flow rate of the gas may be controlled by the rate limiting orifice (gas flow outlet). The therapeutic gas may also be radially diffused as it travels through the nosepiece. Filtering (e.g., of particles settling in the device during the manufacturing process) of the therapeutic gas may also occur as the gas passes through the nosepiece. When flowing through the nosepiece, the flow rate of the therapeutic gas may not be substantially restricted by it. For example, the flow rate of the gas flowing through the nosepiece is reduced by less than about 1% of a desired or predetermined gas flow rate generated by the restrictive orifice.

Methods for using the hand-held, low flow gas dispensing devices to deliver a therapeutic gas, e.g., carbon dioxide, at a controlled and fixed flow rate are also described herein. In general, the method for delivering a therapeutic gas to the nasal mucosa includes inserting a nosepiece of a hand-held, low flow gas dispenser into a nasal cavity, where the nosepiece has a wall comprising a porous material having a pore size; generating a flow of therapeutic gas from a compressed gas cylinder by actuating an activation mechanism; regulating the pressure (e.g., down-regulating the pressure) and controlling the flow of the therapeutic gas released from the compressed gas cylinder using a gas flow outlet (e.g., a restrictive orifice); and diffusing the flow of the therapeutic gas as it passes through the porous material of the nosepiece wall. The step of regulating gas pressure may be accomplished using a pressure regulator having a regulator valve, a diaphragm, and a diaphragm pin assembly. The step of diffusing the flow of the therapeutic gas will generally reduce the sensation of stinging of the nasal mucosa felt by a patient. Diffusion of the therapeutic gas may be adjusted or tailored in any suitable fashion to reduce the stinging of the nasal mucosa during gas delivery. For example, the therapeutic gas may be diffused in a radial pattern, or through selective areas of the nosepiece. However, as previously stated, the nosepiece does not substantially restrict the flow rate of the gas. The method may also include filtering the flow of therapeutic gas it passes through the porous material of the nosepiece wall. Methods for treating medical conditions such as headaches (e.g., migraine headaches, cluster headaches, tension headaches, etc.); allergies (e.g., allergic rhinitis); asthma; and nervous disorders with the therapeutic gas are also described.

Alternatively, methods for delivering a therapeutic gas to the nasal mucosa may include the steps of inserting a nosepiece of a hand-held gas dispenser into a nasal cavity, the nosepiece having a wall comprising a porous material having a pore size, and the gas dispenser comprising a gas control assembly having a pressure regulator and a restrictive orifice; generating a flow of high pressure therapeutic gas from a compressed gas cylinder by actuating an activation mechanism; reducing the pressure of the therapeutic gas; controlling to a predetermined flow rate the rate of flow to the nosepiece of the reduced pressure therapeutic gas; supplying the reduced pressure therapeutic gas to the nosepiece at the predetermined flow rate; and diffusing the flow of the reduced pressure therapeutic gas as it passes through the porous material of the nosepiece wall.

Some methods for delivering a therapeutic gas to a patient's nasal mucosa comprise: inserting a nosepiece of a hand-held, low flow gas dispenser into a nasal cavity, the nosepiece having a wall comprising a porous material having a pore size, and the gas dispenser comprising, a gas control assembly having a pressure regulator and a restrictive orifice gas flow outlet; generating a flow of the therapeutic gas from a compressed gas cylinder by actuating an activation mechanism; using the pressure regulator to reduce the pressure of the generated flow of therapeutic gas; using the restrictive orifice to control to a desired flow rate the rate of flow to the nosepiece of reduced pressure therapeutic gas; supplying therapeutic gas at a reduced pressure and at the desired flow rate to the nosepiece; and diffusing the flow of the therapeutic gas as it passes through the porous material of the nosepiece wall so as to deliver the therapeutic gas to the patient's nasal mucosa substantially at the desired flow rate.

The therapeutic gases may also be used in a method of treating allergy in a patient, the method comprising the steps of inserting a nosepiece of a hand-held gas dispenser into a nasal cavity of the patient, the nosepiece having a wall comprising a porous material; and within the dispenser: generating a flow of high pressure therapeutic gas; reducing the pressure of the generated flow of high pressure therapeutic gas; controlling to a desired flow rate a rate of flow to a dispenser nosepiece of reduced pressure therapeutic gas; supplying reduced pressure therapeutic gas to the nosepiece at said desired flow rate; and diffusing the flow of the therapeutic gas as it passes through the porous material of the nosepiece wall so as to deliver the therapeutic gas to the patient's nasal mucosa substantially at said desired flow rate. The desired flow rate may range from between 0.20 and 1.00 standard liters per minute (SLPM), between 0.35 to 0.65 SLPM, or between 0.40 and 0.60 SLPM. The therapeutic gas for use in a method of treating allergy in a patient may comprise: inserting a nosepiece of a hand-held gas dispenser into a nasal cavity of the patient, the nosepiece having a wall comprising a porous material; and within the dispenser: generating a flow of high pressure therapeutic gas; reducing the pressure of the generated flow of high pressure therapeutic gas; controlling to a desired flow rate a rate of flow to a dispenser nosepiece of reduced pressure therapeutic gas; supplying reduced pressure therapeutic gas to the nosepiece at said desired flow rate; and diffusing the flow of the therapeutic gas as it passes through the porous material of the nosepiece wall so as to deliver the therapeutic gas to the patient's nasal mucosa substantially at said desired flow rate. Here the step of regulating gas pressure (e.g., reducing gas pressure) may be accomplished using a pressure regulator having a regulator valve, a diaphragm, and a diaphragm pin assembly. The step of diffusing the flow of the therapeutic gas will generally reduce the sensation of stinging of the nasal mucosa felt by a patient. Diffusion of the therapeutic gas may be adjusted or tailored in any suitable fashion to reduce the stinging of the nasal mucosa during gas delivery. For example, the therapeutic gas may be diffused in a radial pattern, or through selective areas of the nosepiece. However, as previously stated, the nosepiece does not substantially restrict the flow rate of the gas. The method may also include filtering the flow of therapeutic gas it passes through the porous material of the nosepiece wall.

Methods of assembling a hand-held, low flow gas dispenser for intranasally delivering a therapeutic gas to a patient via a diffusing and filtering nosepiece assembled to a dispenser gas outlet are also described herein. In the methods, the dispenser may generally include a gas control assembly including a pressure regulator for reducing the pressure of gas supplied thereto and a restrictive orifice for controlling the rate of flow of reduced pressure gas supplied thereto by the pressure regulator. Here the method may further include the sequential steps of: adjusting the pressure regulator to provide gas to the dispenser outlet, when the nosepiece is not assembled thereto, at a desired delivery pressure and flow rate; and assembling the nosepiece to the dispenser gas outlet so as to enable gas to be intranasally delivered to the patient substantially at the desired delivery pressure and flow rate via the assembled nosepiece. Thus, a method of assembling a hand-held, low flow gas dispenser for intranasally delivering a therapeutic gas to a patient via a diffusing and filtering nosepiece assembled to a dispenser gas outlet, the dispenser having a gas control assembly including a pressure regulator for reducing the pressure of gas supplied thereto and a restrictive orifice for controlling the rate of flow of reduced pressure gas supplied thereto by the pressure regulator, may comprise the sequential steps of: adjusting the pressure regulator to provide gas to the dispenser outlet, when the nosepiece is not assembled thereto, at a desired delivery pressure and flow rate; and assembling the nosepiece to the dispenser gas outlet so as to enable gas to be intranasally delivered to the patient substantially at the desired delivery pressure and flow rate via the assembled nosepiece. Assembly according to these methods may be useful given that the manner of assembly allows the gas flow rate to be controlled to a desired flow rate, and because the gas at the desired flow rate is automatically diffused by the nosepiece, the gas dispensing devices described herein do not include or require flow adjustment features for manipulation by the patient, and are thus simple to operate.

The methods also generally include the delivery of carbon dioxide and other gases to patients for relieving symptoms associated with headache (e.g., migraine headaches, tension-type headaches, cluster headaches), jaw pain, facial pain (e.g., trigeminal neuralgia), allergies (rhinitis and conjunctivitis), asthma, nervous disorders (e.g., epilepsy, Parkinson's), and other common ailments.

The hand-held devices described here are simple to use and infuse or bathe the mucous membranes of the nasal cavity of a patient with a treatment gas that induces a therapeutic effect/relieves symptoms while reducing the nasal sensation (e.g., stinging) often experienced by the patient. An exemplary treatment gas is carbon dioxide but other gases such as nitric oxide, oxygen, isocapnic mixtures of gaseous acids, helium, and the like, will also find use. The therapeutic gases may be used in a substantially pure form without other gases, active agents, or other substances that dilute the therapeutic gas or that have other biological activities. In other instances, however, the therapeutic gases may be combined with other substances. For example, the therapeutic gases may be combined with other gases, such as inert carrier gases, active gases, solids to form aerosols, liquid droplets to form aerosols or sprays (e.g., the gases may be combined with saline), powders, or the like to potentiate (enhance) their effects. Conversely, these agents combined with the therapeutic gas can potentiate the effects of the therapeutic gas. In such instances, the therapeutic gases and mixtures may have biological activities in addition to the relief of symptoms accompanying common ailments. In all instances, however, the carbon dioxide or other principle therapeutic gas will be delivered in a quantity and over a time course that results in the reduction or elimination of the symptom that is being treated.

The therapeutic gas provides for the desired symptomatic relief by infusing the treatment gas into a nasal cavity while having the patient refrain from inhaling the therapeutic gas. A relatively low volume of the carbon dioxide or other treatment gas can thereby be used to achieve the desired therapeutic effect. In addition, substantial exclusion from the lungs permits the use of the treatment gas at high (chronically unbreathable) concentrations, often being substantially pure approaching 100%, which is necessary to achieve maximum effective treatment via the nasal mucosa. Furthermore, nasal infusion of a chronically unbreathable mixture of an inert carrier gas with nitric oxide permits direct delivery of nitric oxide to the treated mucosa without the oxidation of nitric oxide that would occur if the carrier gas were a chronically breathable mixture of nitric oxide with air or oxygen.

In the case of mild headaches, rhinitis, or similar conditions, a total carbon dioxide volume as low as one cubic centimeter (cc) delivered over a time as short as one second may achieve adequate symptomatic relief. Of course, for more severe symptoms, such as those associated with migraine headache, the total treatment volumes of carbon dioxide and treatment times may be much greater.

The treatment steps may occur as a single infusion or multiple infusions. The length of any particular infusion step may depend, among other things, upon the desired dose to be delivered, or the degree of relief the patient is experiencing, i.e., the patient may continue and/or repeat infusions until relief is achieved. Single infusion steps usually will be performed for a time in the range from about 1 second to about 20 seconds for rhinitis relief and about 1 second to about 60 seconds for headache relief, and more usually from about 2 seconds to about 15 seconds for rhinitis and about 10 seconds to about 30 seconds for headache. The infusing steps may be repeated one, two, three, four, or more times in order to achieve the desired total treatment time.

### EXAMPLES

### Example 1

### Method of Making an Exemplary Diffusing and Filtering Nosepiece

A diffusing and filtering nosepiece may be produced by sintering of one of the polymeric materials described herein to form a porous plastic part. Sintering is a manufacturing process that is used to make porous components from thermoplastic powders or pellets (especially micropellets). In most sintering processes, the powdered material is held in a mold and then heated to a temperature below the melting point. The atoms in the powder or pellet particles diffuse across the boundaries of the particles at each particle-to-particle interface, fusing the particles together at the point of contact but leaving air space in the “gaps”. The result is a cohesive open cell structure with well controlled pore size and pore volume. The typical pore size may be in the range of 5 to 500 microns.

1. A hand-held, low flow gas dispenser for intranasally delivering a therapeutic gas to a patient comprising:
a housing having a distal end and a proximal end;
a cylinder within the housing and having a compressed therapeutic gas contained therein;
a gas control assembly coupled to the cylinder; and
a diffusing and filtering nosepiece attached to the distal end of the housing, the nosepiece having a wall defining a chamber in fluid communication with the gas control assembly, the wall having a wall thickness and an internal surface area, and comprising a porous material having a pore size,
wherein the porous material is configured to diffuse and filter the compressed therapeutic gas as the gas flows through the nosepiece wall, and the gas control assembly comprises a restrictive orifice for controlling the rate of flow of the gas from the cylinder to the nosepiece.

2. The gas dispenser of claim 1, wherein the gas control assembly further comprises a pressure regulator.

3. The gas dispenser of claim 2, wherein the pressure regulator comprises:
a regulator valve;
a diaphragm and a diaphragm pin assembly, the regulator valve being coupled to the diaphragm by the diaphragm pin assembly; and
a regulating spring and an adjustment screw.

4. The gas dispenser of claim 1, wherein the restrictive orifice is disposed proximal to the nosepiece.

5. The gas dispenser of claim 1, wherein the restrictive orifice has a diameter from about 0.015 cm (0.006 in) to about 0.025 cm (0.010 in).

6. The gas dispenser of claim 1, wherein the restrictive orifice has a diameter of about 0.020 cm (0.008 in).

7. The gas dispenser of claim 1, wherein the porous material is selected from the group consisting of sintered ultra high molecular weight polyethylene, polypropylene, polytetrafluoroethylene (PTFE), polytetrafluoroethylene (PTFE), polyvinylidene fluoride (PVDF), ethylene vinyl acetate (EVA), high density polyethylene (HDPE), low density polyethylene (LDPE), very low density polyethylene (VLDPE), polystyrene, polycarbonate (PC) and PC/ABS blends, nylon, polyethersulfone, and combinations thereof.

8. The gas dispenser of claim 1, wherein the porous material comprises sintered ultra high molecular weight polyethylene.

9. The gas dispenser of claim 1, wherein the nosepiece comprises a sintered metal.

10. The gas dispenser of claim 9, wherein the sintered metal comprises stainless steel, nickel, titanium, copper, aluminum, and alloys and combinations thereof.

11. The gas dispenser of claim 1, wherein the pore size ranges from about 10 microns to about 100 microns.

12. The gas dispenser of claim 11, wherein the pore size ranges from about 15 microns to about 50 microns.

13. The gas dispenser of claim 12, wherein the pore size ranges from about 20 microns to about 28 microns.

14. The gas dispenser of claim 1, wherein the nosepiece thickness ranges from about 0.10 cm to about 0.35 cm.

15. The gas dispenser of claim 14, wherein the wall thickness is about 0.17 cm.

16. The gas dispenser of claim 1, wherein the wall thickness of the nosepiece is varied.

17. The gas dispenser of claim 1, wherein the compressed therapeutic gas is selected from the group consisting of carbon dioxide, nitric oxide, oxygen, helium, and combinations thereof.

18. The gas dispenser of claim 1, wherein the compressed therapeutic gas comprises carbon dioxide.

19. A method for delivering a therapeutic gas to the nasal mucosa comprising:
inserting a nosepiece of a hand-held gas dispenser into a nasal cavity, the nosepiece having a wall comprising a porous material having a pore size, and the gas dispenser comprising a gas control assembly having a pressure regulator and a restrictive orifice;
generating a flow of high pressure therapeutic gas from a compressed gas cylinder by actuating an activation mechanism;
reducing the pressure of the therapeutic gas;
controlling to a predetermined flow rate the rate of flow to the nosepiece of the reduced pressure therapeutic gas;
supplying the reduced pressure therapeutic gas to the nosepiece at the predetermined flow rate; and
diffusing the flow of the reduced pressure therapeutic gas as it passes through the porous material of the nosepiece wall.

20. The method of claim 19, wherein the restrictive orifice controls the flow rate of the therapeutic gas generated by the compressed gas cylinder.

21. The method of claim 19, wherein the predetermined flow rate of the therapeutic gas is between about 0.30 SLPM to about 0.70 SLPM.

22. The method of claim 21, wherein the predetermined flow rate of the therapeutic gas is between about 0.40 SLPM to about 0.60 SLPM.

23. The method of claim 22, wherein the predetermined flow rate of the therapeutic gas is between about 0.40 SLPM to about 0.60 SLPM.

24. The method of claim 23, wherein the predetermined flow rate of the therapeutic gas is between about 0.50 SLPM.

25. The method of claim 19, wherein the pressure regulator reduces the pressure of the therapeutic gas flowing through the restrictive orifice.

26. The method of claim 19, wherein the reduced pressure of the therapeutic gas is between about 12 psig to about 20 psig.

27. The method of claim 19, wherein the porous material comprises sintered ultra high molecular weight polyethylene.

28. The method of claim 19, wherein the pore size ranges from about 10 microns to about 100 microns.

29. The method of claim 28, wherein the pore size ranges from about 15 microns to about 50 microns.

30. The method of claim 29, wherein the pore size ranges from about 20 microns to about 28 microns.

31. The method of claim 19, further comprising the step of filtering the flow of the reduced pressure therapeutic gas as it passes through the porous material of the nosepiece wall.

32. The method of claim 19, wherein the flow rate of the therapeutic gas through the nosepiece is reduced by less than 1% of the predetermined flow rate.

33. The method of claim 19, wherein the therapeutic gas is radially diffused as it passes through the nosepiece.

34. The method of claim 19, wherein the therapeutic gas is selected from the group consisting of carbon dioxide, nitric oxide, oxygen, helium, and combinations thereof.

35. The method of claim 19, wherein the therapeutic gas comprises carbon dioxide.

36. The method of claim 19, wherein the therapeutic gas is used to treat a medical condition selected from the group of headaches, allergies, asthma, and nervous disorders.
